(19) United States
(12) Patent Application Publication
(10) Pub. No.: US 2026/0166096 A1
(43) Pub. Date: Jun. 18, 2026

(54) A MULTIFUNCTIONAL VULNERARY COMPOSITION

(71) Applicant: Carlos ALVARADO, Guatemala (GT)

(72) Inventor: Carlos ALVARADO, Guatemala (GT)

(21) Appl. No.: 19/472,543

(22) PCT Filed: Oct. 21, 2023
(86) PCT No.: PCT/IB2023/060644

§ 371 Date: Oct. 3, 2025

(51) Int. Cl. A61K 35/644; A61K 33/30; A61K 36/48; A61P 17/02

(52) U.S. Cl. CPC A61K 35/644; A61K 33/30; A61K 36/48; A61P 17/02; A61K 2236/15; A61K 2236/53

(57) ABSTRACT

The present invention discloses a new multifunctional vulnerary composition, comprising a combination of natural ingredients that work synergistically in the reconstruction of skin tissues in mammals, affected by a variety of injuries or conditions, due to trauma, disease, or surgery. The natural ingredients herein, of bee honey, trigonelline (N-methylnicotinic acid), and zinc oxide, whose simultaneous antioxidant, antimicrobial, anti-inflammatory, and immunomodulatory properties, offer innovative strategies for restorative-regenerative skin injuries. Surprisingly, it has been discovered that by combining honey, trigonelline (N-methylnicotinic acid) and zinc oxide in precise proportions, provide a comprehensive, safe, and effective solution for the repair-regeneration and restructuring of the mammal's skin wounds. By taking advantage of the potential of this composition, health professionals optimize solve the problems of chronic wounds or tissues affected by injuries or conditions in a minimum of time and improve the quality of life of subjects who present these conditions.

### TECHNICAL FIELD

A multifunctional vulnerary composition

### FIELD OF THE INVENTION

The invention relates to a multifunctional vulnerary composition for promoting or expedite tissue repair, regeneration, and restructuring of damaged skin tissues in mammals and preparation procedure useful in skin repair, regeneration, and reconstruction.

### CROSS-REFERENCE TO RELATED APPLICATIONS

“Not Applicable”

### STATEMENT REGARDING FEDERALLY SPONSORED RESEARCH OR DEVELOPMENT

“Not Applicable”

### THE NAMES OF THE PARTIES TO A JOINT RESEARCH AGREEMENT

“Not Applicable”

### REFERENCE TO A “SEQUENCE LISTING

“Not Applicable”

### STATEMENT REGARDING PRIOR DISCLOSURES BY THE INVENTOR OR A JOINT INVENTOR

“Not Applicable”

### BACKGROUND

Chronic wounds are a prevalent and complex healthcare problem affecting millions of individuals worldwide.

These wounds arise from a variety of causes, such as pressure ulcers, diabetic foot ulcers, venous leg ulcers, and arterial ulcers.

The underlying factors that contribute to chronic wounds are multifaceted and interrelated, making their treatment particularly difficult.

Chronic wounds cause a significant burden on individuals and healthcare systems worldwide, due to their delayed healing and associated complications.

Despite the numerous resources available, the factors that contribute to the development and persistence of these lesions remain complex and multifaceted, which carriage significant challenges in the context of repair-regeneration and restructuring of injured tissues.

To enhance comprehension and advance the field of chronic wound research, it is imperative to assess the existing knowledge and identify areas that require further exploration.

Therefore, achieving a comprehensive understanding of these intricate processes is vital to solving the complexities of chronic wounds and developing targeted interventions.

These wounds are characterized by a disrupted healing process that is influenced by various factors within the microenvironment of the wound bed.

Problems that occur in the chronic wound bed (microenvironment) are caused by the individually or in combination issues, characterized by inflammation, infection, oxidation, immune system deficiencies, cellular mechanisms and impaired molecular function, reduced angiogenesis, and granulation tissue formation, as well as continued breakdown and inactivation of the extracellular matrix (ECM).

### Excessive Inflammation

Inflammation is a critical component of the wound healing process.

However, in chronic wounds, inflammation is often prolonged and disordered, affects the balance of cellular and molecular events, further complicating existing tissue damage.

This sustained inflammatory state disrupts the stability between pro-inflammatory and anti-inflammatory signals, impairs cellular activities, impedes the progression toward comprehensive wound healing, and inhibits the transition to the proliferative phase of wound healing.

When the delicate balance between inflammation and healing is disrupted, the body's ability to repair damaged tissues becomes compromised.

Chronic wounds, such as diabetic ulcers, are particularly susceptible to this detrimental cycle of inflammation, leading to non-healing wounds and increased morbidity.

Ingawale et al. (2015)^{[1]} comment in their studies that inflammation and oxidative stress are associated with each other, and include the immune system, in which inflammation is the natural innate response to pathogens.

Information that is consistent with the investigations of Erejuwa et al. (2014)^{[2]} who highlight that the uncontrolled inflammatory processes, together with the presence of oxidative stress, plays a key role in the pathophysiological incidence of chronic disorders such as traumatic and autoimmune diseases.

Studies have shown that sustained inflammation at the wound site prolongs the inflammatory phase, leading to delayed wound closure and increased risk of infection.

Furthermore, the imbalance between pro-inflammatory and anti-inflammatory mediators plays a crucial role in perpetuating chronic inflammation.

In chronic wounds, there is an overabundance of pro-inflammatory cytokines and a deficiency of anti-inflammatory cytokines, disrupting the delicate balance required for proper healing.

Inflammatory mediators play a crucial role in our body's immune response, aiding in the defense against pathogens and facilitating tissue repair.

However, elevated levels of inflammatory mediators can have detrimental effects on the healing process by causing damage to essential growth factors and the extracellular matrix.

### Disruption of Essential Growth Factors

Inflammatory mediators, such as cytokines and chemokines, are responsible for coordinating immune responses and regulating cellular activities.

Unfortunately, excessive, or prolonged inflammation can disrupt the delicate balance of growth factors required for tissue repair.

Growth factors like transforming growth factor-beta (TGF-β) and platelet-derived growth factor (PDGF) are essential for cellular proliferation, angiogenesis, and extracellular matrix deposition.

Nevertheless, the presence of inflammatory mediators can interfere with the signaling pathways involved in growth factor production, leading to impaired healing and delayed tissue regeneration.

Inflammatory mediators, particularly matrix metalloproteinases (MMPs), can be upregulated during inflammation and can cause the degradation of extracellular matrix (ECM) components.

The negative effects of elevated inflammatory mediators on growth factors, extracellular matrix (ECM), and angiogenesis collectively contribute to delayed wound healing.

Inflammatory mediators can also influence the formation of scars. Excessive inflammation during the healing process can result in the overproduction of collagen and fibrous tissue, leading to hypertrophic or keloid scars.

In conclusion, elevated levels of inflammatory mediators can disrupt essential growth factors, degrade the extracellular matrix, impair angiogenesis, delay wound healing, and contribute to increased scar formation.

### Infection

Infection is a significant contributing factor to delayed wound healing, exerting its impact through various mechanisms within the microenvironment of chronic wounds.

According to Wu et al. (2015)^{[3]}, Pseudomonas aeruginosa is a widely distributed type of Gram-negative bacterium responsible for nosocomial infections, acquired in healthcare settings, leading to severe infections in people with compromised immune systems.

In 2017, Azam and Khan (2019)^{[4]}, described P. aeruginosa as one of the most difficult bacteria, causing a significant threat to human life, recognized by the World Health Organization (WHO) acknowledged its severity and designated it as a priority pathogen for intensive research and the development of new antibiotics.

The key aspects associated with infection, and the overall microenvironment in chronic wound, including:

### Infection and Inflammation

Infection triggers a cascade of events that lead to inflammation within the microenvironment of chronic wounds.

This process involves the release of pro-inflammatory molecules, such as cytokines (e.g., interleukins) and tumor necrosis factor-alpha (TNF-α).

The presence of pathogens and their byproducts stimulates immune cells, causing an arrival of neutrophils and macrophages to the wound site.

Consequently, inflammation becomes a characteristic feature of chronic wound infections.

Furthermore, infection within chronic wounds affects the delicate balance of pro-angiogenic and anti-angiogenic factors, leading to impaired angiogenesis, that is critical for wound healing as it supplies oxygen, nutrients, growth factors, and immune cells to the wound site.

The insufficient blood supply compromises the delivery of necessary components because infection profoundly affects the microenvironment of chronic wounds, conducting to delayed wound healing.

### Immune System and Infection

The immune system plays a crucial role in combating infections.

Neutrophils, macrophages, and other immune cells are mobilized to the wound site to eliminate pathogens.

However, in chronic wounds, the immune response can become dysregulated, compromising the clearance of pathogens, and prolonging the inflammatory state.

Persistent infection weakens the immune system's ability to clear pathogens efficiently, leading to a prolonged inflammatory state and impairing the healing process.

In a publication by Kirchner et al. (2020)^{[5]}, it is noted that the skin, in its remarkable capacity to repair and protect wounds, promotes the formation of an immunological microenvironment within and around the affected tissue.

This microenvironment, the authors describe, encompasses multiple innate and adaptive processes, including the recruitment of immune cells to the wound site and the release of extracellular factors that directly facilitate wound healing and strengthen defenses against microbial threats to the wound.

### Microenvironment of Chronic Wounds and Infection

The process of wound healing is a comprehensive reaction to injury that impacts the entire body, rather than just the specific area of tissue damage.

It has become evident that the wound microenvironment plays a vital role in this regard.

Comprehending the intricate functionality of the wound microenvironment provides insights into how different factors influence or hinder the normal healing processes.

Factors such as decreased oxygenation, increased alkalinity, and infection contribute to an unfavorable milieu for effective wound healing.

In the microenvironment the presence of local cells, such as fibroblasts, play a crucial role in wound healing by synthesizing the extracellular matrix (ECM).

However, infection can impair the functionality and guidance it provides of these cells, leading to a compromised extracellular matrix (ECM) synthesis.

### Oxidation and Reactive Oxygen Species (ROS)

Chronic wounds are characterized by increased oxidative stress and the accumulation of reactive oxygen species (ROS).

This oxidative environment is detrimental to cellular processes and impairs wound healing.

ROS disrupts cell signaling pathways, impairs migration and proliferation of key cells involved in wound healing (fibroblasts and keratinocytes), and leads to DNA damage, protein degradation, and lipid peroxidation.

### Impaired Cellular and Molecular Mechanisms

Chronic wounds exhibit deficiencies in cell migration, proliferation, and differentiation.

The impaired function of fibroblasts, keratinocytes, and endothelial cells impedes the formation of granulation tissue and re-epithelialization.

These cells fail to produce essential growth factors, cytokines, and extracellular matrix components necessary for proper wound healing.

### Reduced Angiogenesis and Granulation Tissue Formation

Angiogenesis, the formation of new blood vessels, is a critical process in wound healing that supplies oxygen and nutrients to the healing tissue.

Prolonged inflammation affects angiogenesis by altering the balance between pro-angiogenic and anti-angiogenic factors, and in the latter case, inflammatory mediators promote the release of anti-angiogenic factors, inhibiting blood vessel formation and compromising the delivery of essential nutrients and growth factors to the injured area.

Moreover, chronic wounds are often colonized by biofilms, which are complex communities of bacteria embedded in a protective extracellular matrix, that resist the immune system's efforts and conventional antimicrobial treatments, causing persistent infection and delaying wound healing.

### Extracellular Matrix Alterations

The extracellular matrix (ECM) provides structural support and guidance for cells involved in wound healing, and infection alters the composition and organization of the ECM, compromising its integrity.

This disruption impedes cell migration, angiogenesis, and interferes with the deposition of new collagen.

Consequently, the formation of granulation tissue and re-epithelialization, essential steps in wound closure, are delayed.

### Insufficient Granulation Tissue Formation

Granulation tissue serves as a foundation for wound healing, facilitating the formation of new skin and tissues.

In chronic wounds, the formation of granulation tissue is often inadequate, impairing the closure of the wound.

Conventional resources do not have the ability to promote robust granulation tissue formation, leading to suboptimal outcomes.

This problem arises due to the improper regulation of various essential elements involved in the normal tissue repair response, namely inflammation, angiogenesis, matrix deposition, and cell recruitment.

Typically, the failure of these cellular processes can be attributed to an underlying clinical condition such as vascular disease, diabetes, or the natural aging process, all of which are commonly associated with impaired healing.

The induction of inflammation, dysregulation of the immune response, impairment of local cell function, alterations in the extracellular matrix, and impaired angiogenesis all contribute to the challenges faced in healing chronic wounds.

Understanding these negative aspects is crucial for developing targeted therapies and interventions to promote efficient tissue repair.

While significant progress has been made in understanding and addressing the challenges in the chronic wound microenvironment, research and innovation into new alternatives is imperative to develop effective solutions for people with chronic wounds.

As previously explained, chronic wounds pose significant challenges in the field of wound healing, which require comprehensive approaches that address the underlying cellular, molecular, and local factors that contribute to their resolutions.

Understanding impaired cellular responses, dysregulated molecular signaling pathways, and local factors within the wound microenvironment is vital to developing effective strategies in the management and healing of chronic wounds.

In recent years, substantial efforts have been made to develop solutions to address the challenges in the chronic wound microenvironment, which are explored below.

### Solutions for Inflammation and Infection

To combat chronic wound inflammation, advanced wound dressings have been developed with anti-inflammatory properties.

These dressings incorporate substances like silver, honey, or Poly hexamethylene biguanide (PHMB), which have antimicrobial effects and promote a controlled inflammatory response.

Furthermore, targeted antimicrobial therapies, including antimicrobial peptides, biofilm-disrupting agents, and novel antibiotic formulations, have shown promise in challenging persistent infection in chronic wounds.

According to Berkes and Monsul, as referenced in the United States Patent Application US20140037688-A1^{[6]}, the successful treatment of chronic infections or chronic biofilm-associated inflammatory diseases often requires specific strategies.

These strategies involve targeting and weakening the bacterial biofilm matrix, disrupting the quorum mechanisms that sustain the bacterial community, and enhancing the local innate immunity of the host.

Penetrating or dispersing the protective barrier of the bacterial biofilm, often referred to as the “armor,” plays a crucial role in combating the persistent inflammation caused by biofilm formation.

In the field of modern medicine, the battle against bacterial infections continues, particularly related to the formation of complex communities of microorganisms enclosed in a protective matrix, recognized as bacterial biofilms.

These biofilms allow bacteria to survive in disturbing environments and resist antibiotic treatments, leading to chronic infections and impeding progress in healing chronic wounds.

Biofilm formation is a complex process orchestrated by bacteria to ensure their survival and resistance to eradication efforts.

However, recent research has illuminated the role of plant-derived biofilm inhibitors in disrupting this intricate structure formation.

### Management of Oxidative Stress

The fundamental elements to achieve optimal tissue repair and regeneration are actively functioning cells.

At all stages of the tissue repair and regeneration process, the initial priority is to rapidly initiate cellular repair activity.

This involves rapidly removing active oxygen radicals from the wound bed with products that have antioxidant effects, to minimize the damage caused by active oxygen radicals.

### Improve the Immune Response

Various immunomodulatory agents, such as growth factors (e.g., platelet-derived growth factor, transforming growth factor beta), have been explored to promote a balanced immune response and modulate the inflammatory state.

In addition, cell therapies involving the application of mesenchymal stem cells or immune cells have shown potential to improve immune function and accelerate chronic wound healing.

In a study published by Djoko et al. in 2015^{[7]} it is mentioned that when the organism faces an infection, the first line of defense is formed by the epithelial cells located in the infection sites, and in the case of damage to these cells, a rapid response is produced, which implies the recruitment of specialized immune cells such as neutrophils and macrophages.

According to these authors, these immune cells play a crucial role in suppressing the initial progression of infection, which engulfs invading pathogens through a process known as phagocytosis.

This action then triggers the activation of adaptive immunity, leading to a coordinated response that ultimately results in complete resolution of the infection.

### Restoration of Cellular and Molecular Mechanisms

Advances in tissue engineering and regenerative medicine have generated innovative therapies to restore damaged cellular and molecular mechanisms.

Strategies such as the use of tissue-engineered skin substitutes, growth factor therapies, and gene therapies aim to promote cell migration, proliferation, and differentiation.

These approaches provide a supportive environment for functional tissue regeneration and enhance the overall healing process.

Stimulation of angiogenesis and the formation of granulation tissue

To enhance angiogenesis and granulation tissue formation in chronic wounds, several therapeutic options have emerged.

Growth factors, such as vascular endothelial growth factor (VEGF) and basic fibroblast growth factor (bFGF), which are incorporated into dressings or given by injection to stimulate blood vessel formation and promote the development of granulation tissue.

In addition, techniques such as low-frequency ultrasound, negative pressure wound therapy, and electrical stimulation have been used in promoting angiogenesis and tissue regeneration.

### Promotion of the Formation and Remodeling of ECM

Efforts to address the continued breakdown and inactivation of the extracellular matrix (ECM) has focused on promoting the formation and remodeling of it.

Advanced dressings containing ECM components such as collagen, hyaluronic acid or fibronectin provide a scaffold for cell migration and support the synthesis of this tissue component.

Furthermore, therapies involving the application of matrix metalloproteinase inhibitors or the use of bioactive molecules that promote extracellular matrix (ECM) deposition and remodeling have shown potential to improve wound healing outcomes.

Significant progress has been made in developing solutions to address the challenges present in the microenvironment of chronic wounds.

Advancements in wound dressings, targeted antimicrobial therapies, antioxidant strategies, immunomodulatory agents, cellular and molecular therapies, angiogenic stimulators, and ECM-based approaches offer promising avenues to enhance healing outcomes.

While these resources have their merits in targeting specific issues, they fall short when it comes to comprehensive and simultaneous treatment of chronic wounds.

Exclusively addressing one factor in chronic wound management poses limitations, as it does not consider the complex interplay of multiple factors presents in the chronic wound bed, and the overall healing outcome is expected to be suboptimal.

Addressing only one aspect prevents solving the set of factors that contribute to the persistence of chronic wounds.

To facilitate a synchronized and balanced curative response, it is necessary to provide a comprehensive multi-component therapeutic solution that addresses inflammation, infection, oxidation, immune system deficiencies, cellular and molecular mechanisms, angiogenesis, tissue formation of granulation and the dynamics of the extracellular matrix (ECM).

This approach ensures a holistic and integrated solution to simultaneously address the complexities of chronic wounds and promote effective wound healing.

Understanding the failure of different wound healing resources has led the author to explore innovative approaches and specify the path towards comprehensive healing to improve the quality of life in people suffering from chronic wounds.

In recent years, scientific research has focused on finding and exploring the medical potential of natural products for human health.

In studies previously carried out by the author, the combination of bee honey and trigonelline has shown to be successful in the healing of wounds and extensive burns.

The present invention closely conforms to the progress of a previously studied dressing, referring to the International Patent Publications No. WO 2014041440 A1 Publication date Mar. 20, 2014^{[8]} and Multifunctional-reinforced dressing Inventor Carlos A. Alvarado 2018 Feb. 8, Publication of WO 2018025060 A1^{[9]}.

The patent publication WO 2014041440 A1 disclose a dressing that contains the following components: bee honey, Trigonella foenum graecum extract, policresulen, acetic acid and agar-agar as the gelling agent.

The dressing is used for healing wounds and burns, and include in every single dressing the products and following amounts: Honey of bees: 30.66 g, aqueous extract seed from Trigonella foenum-graecum 7.34 g, Policresulen 120,478 mg and gelling agent Agar-Agar 0.522 g. The procedure also adding Policresulen at 40% with molecular formula: C23H24O12S3 (2-hydroxy-3,5-bis[(4-hydroxy-2-methyl-5-sulfophenyl)methyl]-4-methylbenzenesulfonic Acid) to the dressing to regulate bacterial growth and promote keratoplasty and keratolytic actions at the site of administration comprising inclusion in an effective amount (120,478 mg per dressing) to reduce and maintain the pH in the acidic range (pH 3.10-3.12 approximately).

The process for the preparation of solid seed extract of Trigonella foenum-graecum comprising the steps: A. addition of the components in matrix ball. B. stirring to homogenize mixture C. leaving to macerate for 14 days under refrigeration (at 4° C.) E. filtering the extract in mesh No. 12 for separating seed and F. physical analysis of the extract.

The patent publication WO 2018025060 A1 relates to a surgical or medical dressing which comprises a supporting and reinforcing layer of elastomeric material (as polyethylene homopolymer) embedded in the composition of honey/additives, to supplement the mechanical strength, which would be fabricated as a single integral piece, intended to provide multi-functional properties in the wound environment, particularly for promote or expedite the regeneration or repair deep tissue injuries or chronic wounds such as ulcers.

Several additional characteristics of this new dressing provide an advantage to any wound care product developed from it.

First, the biomaterial retains many biological properties of its major constituents (honey/additives).

Second, the application of this safety dressing is greatly simplified particularly for pediatric burn Care.

The honey/additives composition comprising highly purified substances, which include in every single dressing the products and following amounts: Honeybees: 30.66 g, aqueous extract seed from Trigonella foenum-graecum 7.34 g, Policresulen 120,478 mg and gelling agent Agar-Agar 0.522 g.

With respect to the honey, focusing on various aspects of its practical applications, is provide in large number of patents granted, including the following: EP 0909557 A1, US20030136274, US20040054313, US 20040121020, US20040127826, US20040127826, US20040127837, US 20040131693, US20040258765, US20050033213, US20050255166, US 20060099166, US20080125617, US20090012440, US20100049262, US20100233283, US20140127283, US20140142522, US20150080815, US2015015093, U.S. Pat. Nos. 3,767,784, 5,785,972, 5,980,875, 6,956,144, U.S. Pat. Nos. 7,563,224, 8,303,551, 8,568,790, 8,632,810, 8,815,298, 9,044,489, 9,180,219, USD 745690, USRE 42755, WO 1999055349 A1, WO 2001041776 A2, WO 2001041776 A2, WO 2001067888 A1, WO 2002000269 A1, WO 2002030467 A2, WO 2002087644 A1, WO 2003005978 A1, WO 2003047642 A1, WO 2004000339 A1, WO 2005000032 A1, WO 2005077402 A1, WO 2007009185 A1, WO 2007030023 A1, WO 2007048193 A1, WO 2007137369 A1, WO 2008049578 A2, WO 2011046454 A1, WO 2012134770 A1, WO 2015110552 A1, WO 2016007776 A1, WO 2009147402 A3.

The following patents have recently been published, which include honey, the first of which is the invention patent US20160220722A1 2016 Jan. 29 2019 Dec. 10 Wardell M. and Sabacinski K. with the title of Wound Healing Compositions Involving Medicinal Honey, Mineral ions, and Methylglyoxal, and Methods of Use, including additionally 1.5-75 parts of magnesium ions, 0.5-75 parts of potassium ions, 0.001-10 parts of calcium ions, 0.0001-10 parts of zinc ions, up to 5 parts of rubidium ions, 0.001-20 parts of bromide ions, and up to 20 parts of sulfate ions, and more recently patent WO 2019040185 A1 2019 Feb. 28 by Sabacinski K. and Kaufman J. L. Buckwheat honey and povidone-iodine wound healing dressing, all of these applications are incorporated by reference herein in the Patent Citations.

The same author, Sabacinski K., and Kaufman J. L., introduce the US 20180236009 A1 Buckwheat honey and bacitracin wound-healing dressing, as reference.

With respect to the honey, many recent studies demonstrate the effectiveness of honey in wound healing and Tashkandi (2021)^{[10]} and other authors as: Almasaudi et al. (2016)^{[11]}, and the brilliant works of Peter Molan (2002)^{[12]}, (2006)^{[13]}, (2009)^{[14]}, are also highlighted in multiple research papers produces a wide range of publications.

Based on previous patents, the author set out to find a new way of making the formulation mentioned in those patents.

The main goal was to make it possible to use the formulation in different ways for treating skin problems in newborns and older adults.

After researching for two years, the author successfully created a paste, which has resulted in the preparation of the present patent for the invention.

It is noted that for two years, the author's son used this paste on his one-year-old daughter to prevent and treat diaper-related skin problems, whose observations during this period allowed the formulation to be perfected.

In another case, a colleague of the inventor tested the paste on a severe skin lesion that had caused extensive tissue loss and deep ulceration, and surprisingly and unexpectedly the lesion closed completely.

The important thing to highlight is that the paste wasn't just made by mixing different products together.

The research focused on improving the paste itself.

This was done in a way that wouldn't harm the sensitive skin of both babies and the elderly.

The positive outcomes of using the paste became evident in every case.

The author continues to conduct research to develop wound healing strategies that align with the principles of wide-ranging and synchronized treatment and now present a novel approach that enables comprehensive and simultaneous treatment, leading to improved outcomes for patients suffering from chronic wounds.

A new research strategy involves the development of multifunctional compositions to support innate healing and combat numerous problems that affect incompletely healing lesions, such as extreme inflammation, ischemic damage, scarring, and wound infection.

By integrating infection control, inflammation modulation, oxidation conversion, immune stimulation, ECM reactivation, microenvironment optimization, restoration of cellular and molecular mechanisms, promotion of angiogenesis, and enhancement of formation of granulation tissue, the new composition offers a transformative and comprehensive solution for patients suffering from chronic wounds.

In the multifunctional vulnerary composition, derived from bee honey, seed extract of trigonelline, and zinc oxide, the bioactivities, and antibacterial, anti-inflammatory and antioxidant properties, turn out to be efficient alternatives in the management of multiple skin conditions.

The combination of bee honey, seed extract of trigonelline extract and zinc oxide, induce tissue repair and regeneration through multiple related mechanisms, whose molecular mutually reinforcing effects increase the general effectiveness of healing observed during its use.

The author of the present invention considers that due to the intrinsic properties of this biomaterial, when being used and interact within biological systems, damage to the host's tissues is avoided, attributed in part to the fact that come from nature and the delicate processes of improvement during their elaboration.

To understand the multidimensionality of the effects of this innovation, we first describe the individual actions of each component, and then the integrated actions of the three components, unexpectedly transforming chronic wound care.

Exploration of these elements opens the way for innovative interventions aimed at improving the lives of people affected by wounds.

The bee honey, zinc oxide, and trigonelline has been the focal point of various scientific articles and invention patents due to its potential health benefits.

### Honey for Wound Healing

Honey has gained popularity in recent years as a complementary medicine for various purposes, including wound healing.

Throughout history, honey has been honored for its remarkable medicinal properties and therapeutic benefits, and recent studies have revealed its potential in accelerating wound healing.

The various compounds present in honey contribute to its wound healing properties.

Research has shown that honey can promote wound closure, reduce inflammation, and improve tissue regeneration.

### Bioactive Compounds in Bee Honey

Beyond its basic composition, bee honey is rich in bioactive compounds that confer its therapeutic properties that include enzymes, flavonoids, phenolic acids, and other phytochemicals.

### Enzymes

One of the most notable enzymes present in honey is glucose oxidase. When honey encounters wound exudate, glucose oxidase catalyzes the conversion of glucose into gluconic acid and hydrogen peroxide.

This reaction yields antimicrobial properties, creating an environment unfavorable for bacterial growth.

The release of hydrogen peroxide helps cleanse the wound and prevent infection.

### Flavonoids and Phenolic Acids

Flavonoids and phenolic acids found in bee honey exhibit strong antioxidant and anti-inflammatory effects.

These compounds scavenge free radicals, reducing oxidative stress and minimizing tissue damage.

By mitigating inflammation, honey promotes a favorable environment for wound healing to occur.

### Other Phytochemicals

Bee honey contains a diverse array of phytochemicals that contribute to its healing properties.

These include organic acids, amino acids, and small amounts of plant pigments.

Although present in lower concentrations, these compounds play a role in supporting various cellular processes involved in wound repair and regeneration.

### Anti-Inflammatory Properties

Chronic inflammation can impede the wound healing process, prolonging recovery time and bee honey, with its anti-inflammatory properties, offers a natural solution to counteract this challenge.

The combined effects of honey's bioactive compounds work synergistically to reduce inflammation by inhibiting pro-inflammatory mediators and promoting the release of anti-inflammatory factors.

Honey has been shown to exert anti-inflammatory effects by reducing the production of cytokines and other inflammatory mediators.

The anti-inflammatory effects of honey are attributed to its phenolic compounds, which act as scavengers of free radicals and protect against oxidative stress.

This may help to reduce inflammation reducing swelling and promoting a balanced inflammatory response that contribute to a favorable wound healing environment.

By minimizing inflammation, honey reduces pain and discomfort associated with wounds, facilitating a more comfortable healing experience for patients.

### Antimicrobial Properties

One of the key properties of bee honey that contributes to its wound healing effects is its broad-spectrum antimicrobial activity.

The high sugar content of honey creates a hypertonic environment, drawing moisture out of bacterial cells and inhibiting their growth.

Clinical studies have reported a significant reduction in bacterial load and infection rates in wounds treated with honey-based dressings, attributed to its low pH, high sugar content, the release of hydrogen peroxide by the enzyme glucose oxidase, and content of natural compounds such as Flavonoids.

Its antimicrobial properties help reduce bacterial load, while its enzymatic activity helps debride dead tissue, allowing it to penetrate the wound bed, delivering its therapeutic benefits directly to the injury site.

### Antioxidants Properties

It contains antioxidants, such as phenolic compounds, flavonoids, and vitamin C, which scavenge free radicals and protect against oxidative stress.

These compounds help to protect the wound area from infection by inhibiting the growth of bacteria and scavenging free radicals.

### Modulation of the Immune Response

A balanced immune response is crucial for effective wound healing.

Bee honey exhibits immunomodulatory effects by influencing immune cell activity and cytokine production, which regulates the immune response, preventing excessive inflammatory reaction while promoting the release of growth factors that support tissue regeneration.

Its ability to modulate the immune response and promote tissue regeneration makes it an invaluable resource in the repair-regeneration and restructuring of skin wounds.

### Tissue Regeneration

Honey has been shown to promote the proliferation of fibroblasts and keratinocytes, which are crucial in the process of tissue regeneration.

Furthermore, honey stimulates the release of growth factors, such as transforming growth factor-beta (TGF-β), which plays a pivotal role in wound healing.

TGF-β promotes cell migration, proliferation, and extracellular matrix formation, thus enhancing tissue regeneration.

In a publication by Scepankova et al. (2021)^{[15]}, the benefits of using honey in wound healing are described, including accelerating the growth of new epithelial cells, stimulating the formation of new blood vessels, improving the immune response, and mitigating the risk of infection by harmful microorganisms, which contributes to all stages of wound healing.

Furthermore, honey's ability to promote a moist wound environment has been attributed to its osmotic properties and high viscosity.

Maintaining a moist environment is crucial for wound healing, as it promotes cell migration, angiogenesis, the deposition of granulation tissue, which allows the formation of new and healthy tissue, and facilitates re-epithelialization.

By integrating the knowledge gained from clinical studies, healthcare practitioners can employ the healing power of bee honey to promote optimal wound healing outcomes and enhance patient well-being.

Hydrogel dressings were investigated for healing of skin graft donor sites in patients at the Hospital Universiti Sains Malaysia over a period of 18 months by Imram et al. (2011)^{[16]}.

Thirty-five patients received hydrogel-shaped honey dressings to their graft donor sites.

All donor sites were inspected on days 10, 15, and 20 postoperatively.

Complete healing was observed between day 10 and day 15 postoperatively, with minimal pain, discomfort, or pruritus.

Honey-based hydrogel may be considered effective in the treatment of skin graft donor sites, warranting further study to compare it with existing dressings.

### Trigonelline (N-Methylnicotinic Acid)

Trigonelline is an herbaceous plant native to the Mediterranean region and Western Asia, rich source of bioactive compounds that contribute to its therapeutic potential.

These include saponins, alkaloids, flavonoids, and various other phytochemicals.

### Saponins

Saponins found in Trigonelline exhibit a range of beneficial effects, including anti-inflammatory, antimicrobial, and antioxidant properties.

These compounds help reduce inflammation at the wound site, minimize the risk of infection, and neutralize harmful free radicals that impede the healing process.

### Alkaloids

Trigonelline contains alkaloids such as trigonelline and Gentianine (Gentianineis a pyridine-derived alkaloid), which have been shown to possess anti-inflammatory and wound healing properties.

Trigonelline exhibits antioxidant activity and promotes collagen synthesis, a crucial process for tissue repair, and these alkaloids contribute to facilitate wound closure and stimulate tissue regeneration.

### Flavonoids

The flavonoids found in Trigonelline, such as quercetin (polyphenolic chemical substructure) and rutin (rutinoside or rutinoside), have antioxidant effects that stops oxidation by acting as a scavenger of free radicals, anti-inflammatory, and fight dysfunction endothelium, thus protecting the blood vessels.

By modulating the inflammatory response, flavonoids in Trigonelline help regulate the immune system's activity during the healing process.

### Modulation of Immune Response

A well-regulated immune response is essential for efficient wound healing and Trigonelline demonstrates immunomodulatory effects by influencing the activity of immune cells and cytokine production.

Studies have shown that Trigonelline extracts can modulate the immune response, reducing excessive inflammation while promoting the release of growth factors that support tissue regeneration.

### Collagen Synthesis and Angiogenesis

Trigonelline plays an important role in promoting the synthesis of collagen, which provides structural integrity and strength to newly formed tissue, which is a fundamental process for tissue repair.

Research has demonstrated that Trigonelline extracts stimulate collagen production, enhancing wound closure and facilitating the maturation of granulation tissue.

Additionally, exhibits angiogenic properties, promoting the formation of new blood vessels in the wounds, vital for providing oxygen, nutrients, and immune cells to the site of injury, expediting the healing process.

In conclusion, Trigonelline (N-methylnicotinic acid) emerges as a powerful natural solution in the restructuring and repair-regeneration of skin wounds.

Its bioactive compounds, including saponins, alkaloids, and flavonoids, contribute to its multifaceted healing properties.

By promoting collagen synthesis, angiogenesis, and modulating the immune response, Trigonelline facilitates wound closure, tissue regeneration, and a balanced healing process.

It is noted that Juan Miguel Garcia Gilabert in his invention patent US20150056175-A1^{[17]}, granted on 2015 Feb. 26, with the title Composition to prevent and/or treat dermatosis and method to obtain it, mentions the use of honey from bees and fenugreek (Trigonella foenum-graecum), mixed with 23 natural products and with stevia and lanolin, in its abstract and claim 1, mentions the use of this composition to prevent and/or treat dermatitis, particularly psoriasis, which is far from the indications of the present application, directed to the treatment of chronic wounds of difficult healing.

Studies at the Faculty of Chemical Sciences of the University of San Carlos de Guatemala (1992)^{[18]}, by doctors Chang Espino, M. J. and Navas Escobedo I., carried out a study entitled Evaluation of the healing effect of Trigonella foenum-graecum L. (fenugreek) seeds and Plantago major L. (plantain) leaves on wounds produced in albino rats, making the following observation and conclusion: “both fenugreek seeds and plantain leaves have an action that favors the regeneration of epithelial tissue, thus contributing to the healing process of superficial wounds, and they conclude that no adverse reaction was observed in the study of Trigonella foenum-graecum L. (fenugreek) and Plantago major L. (plantain), when administered daily in topical form.”

Mustafa et al. (2015)^{[19]}, investigated to assess the wound-healing properties of fenugreek seeds on skin excision wounds in male Sprague Dawley rats.

The study employed three distinct cohorts of rats, with each group comprising 6 animals.

In Group 1, the wound sites of the animals were subjected to topical application of pure unboiled honey. For Group 2, a combination approach involving the application of honey along with fenugreek seed extract was employed. Group 3, serving as the control, witnessed the topical administration of Solcoseryl gel onto the wounds.

Throughout the duration of the experiment, the wound sites across all animal groups exhibited a state of cleanliness and sustained sterility.

Comparative analysis revealed noteworthy findings. Both the wounds treated with the combination of honey and fenugreek seed extract, as well as those treated with Solcoseryl gel, displayed a notable acceleration in the wound healing process (p<0.05) as opposed to wounds treated solely with honey.

Evidenced by these outcomes, it is evident that the incorporation of fenugreek seed extract holds substantial potential in expediting wound healing processes and elevating wound sterility rates. The observations of this study contribute to the mounting body of evidence substantiating the beneficial impacts of fenugreek seed extract on wound healing dynamics.

### Zinc Oxide

Zinc oxide is a mineral that has been used for medicinal purposes for centuries, to treat various skin conditions, including wounds.

### Properties and Effectiveness of Zinc Oxide

Zinc oxide it has both antimicrobial and anti-inflammatory properties, which make it an effective agent against wound infections.

Its effectiveness in wound healing has been attributed to its ability to increase epithelialization, neovascularization, and collagen synthesis.

### Mechanisms of Zinc Oxide's Wound Healing Effects

Zinc oxide exerts its wound healing properties through several different mechanisms.

One of the key properties of zinc oxide is its antimicrobial activity.

It acts as a physical barrier, preventing the entry of microorganisms into the wound and reducing the risk of infection.

Zinc oxide also has a drying effect, which helps absorb excess moisture from the wound, creating a conducive environment for healing.

In addition to its antimicrobial properties, zinc oxide has anti-inflammatory effects.

It helps reduce inflammation by inhibiting the release of pro-inflammatory cytokines and modulating immune cell activity, that contribute to its wound healing effects.

By reducing inflammation at the wound site, zinc oxide helps create an optimal healing environment by modulating the release of pro-inflammatory cytokines and promoting the production of anti-inflammatory mediators.

This anti-inflammatory effect aids in minimizing tissue damage and supporting the progression of the healing cascade.

Zinc oxide also plays a role in tissue regeneration and wound closure.

It stimulates epithelial cell proliferation and migration, leading to the formation of a new layer of skin over the wound.

It has been shown to enhance the proliferation and migration of cells involved in wound healing, such as fibroblasts and keratinocytes.

It also promotes angiogenesis, or the formation of new blood vessels, which is necessary for the growth of new tissue.

Furthermore, zinc oxide promotes collagen synthesis and deposition, contributing to the strength and integrity of the healing tissue.

According to a report by Lansdown et al. (2007)^{[20]}, zinc is an essential trace element in the human body and plays a vital role as a cofactor in various transcription factors and enzyme systems.

One such system is the zinc-dependent matrix metalloproteinases, which contribute to debridement and the migration of keratinocytes during wound repair.

The authors provide a comprehensive account of zinc's role in wound management, specifically in relation to current concepts of wound bed preparation, debridement, anti-infective action, and promotion of epithelialization.

In their research, the authors highlight the following significant aspects regarding zinc's impact on wound healing:

Zinc exhibits beneficial properties in reducing superinfections and eliminating necrotic material.

It does so by enhancing local defense systems and promoting collagenolytic activity.

Ravanti and Kähäri (2000)^{[21]} refer to the fact that the addition of supplementary zinc ions can expedite many biochemical and molecular events in wound repair.

This is achieved through the up regulation of Metallothioneins (MTs) and zinc metalloenzymes.

To address the above issues, attention has been paid to those compounds capable of actively stimulating the healing-related functions of cells, which in addition to utilizing the synergy of multifunctional physicochemical properties and biological activities of them, and possessing other functions, such as anti-inflammatory, antibacterial or antioxidant.

In the context of skin wounds, the integration of bee honey, trigonelline, and zinc oxide, as it has been described in the lines above in the individual actions of each one of them, plays a significant role in optimizing the repair-regeneration and restructuring process.

Combining the powers of three components, bee honey, trigonelline, and zinc oxide, demonstrates a synergistic effect in wound restructuring.

The integration of these three components creates a harmonious unite that addresses multiple aspects of wound healing, enhancing the overall regenerative process.

These products exhibit anti-inflammatory, antimicrobial, antioxidant, immunomodulatory actions, positive effects on the wound microenvironment, extracellular matrix restructuring, and promotion of angiogenesis.

### Anti-Inflammatory Action

They actively inhibit the release of inflammatory mediators and reduce excessive inflammatory responses.

By creating an optimal environment for tissue repair and regeneration, the composition expedites the healing process.

### Antimicrobial Action

The presence of microorganisms in a wound can inhibit healing and lead to complications.

However, the antimicrobial properties of the composition (bee honey, trigonelline, and zinc oxide) aid in combating infections and reducing bacterial burden at the wound site.

Through inhibiting the growth of bacteria and other pathogens, the composition establishes a more conducive environment for tissue repair.

### Antioxidant Action

Oxidative stress can delay the healing process.

However, the composition (bee honey, trigonelline, and zinc oxide) neutralizes free radicals and mitigates oxidative stress.

This protection safeguards cells and tissues from oxidative damage, facilitating faster and more effective healing.

### Immunomodulatory Action

The immune system plays a crucial role in wound healing.

The composition exhibits immunomodulatory effects, regulating and balancing the immune response.

By enhancing the body's defense mechanisms, these compositions promote immune cell activity and facilitate pathogen clearance. Simultaneously, they reduce excessive inflammation, contributing to accelerated healing.

### Positive Effects on the Wound Microenvironment

The microenvironment of a wound is pivotal in the healing process.

The composition creates a favorable environment at the wound site by providing essential nutrients and growth factors necessary for cellular proliferation and tissue formation.

They also promote proper tissue oxygenation, improving cell viability and supporting efficient healing.

Additionally, they help maintain an optimal moisture balance in the wound, crucial for clot formation and tissue regeneration.

### Restructuring of the Extracellular Matrix

The extracellular matrix is a three-dimensional framework that provides structural support to tissues.

During the healing process, the matrix undergoes restructuring to enable cellular migration and proliferation.

The composition (bee honey, trigonelline, and zinc oxide) stimulates the synthesis and deposition of key matrix components such as collagen and elastin.

This strengthens damaged tissues and enhances their structural integrity, essential for proper healing.

### Promotion of Angiogenesis

Angiogenesis, the formation of new blood vessels from existing ones, is critical for wound healing.

The composition promotes angiogenesis by stimulating the release of growth factors and the proliferation of endothelial cells.

This results in improved vascularization at the wound site, expediting the healing process.

### Synergistic Actions and Reinforcement of Effects

When these three products act in combination, synergistic actions are established, amplifying their individual effects on tissue repair, regeneration, and restructuring.

The combination of their anti-inflammatory, antimicrobial, antioxidant, immunomodulatory, positive wound microenvironment, extracellular matrix restructuring, and angiogenesis-promoting properties creates an optimal environment for healing.

The interactions between these products potentiate their individual benefits, producing greater efficacy in the healing process.

Their synergistic action contributes to the reduction of inflammation, control of bacterial load, cellular protection against oxidative stress, modulation of the immune response, and promotion of tissue formation.

This approach addresses multiple aspects of the healing process, resulting in accelerated and improved recovery.

In conclusion, the combination of these three products offers a remarkable solution for promoting tissue repair, regeneration, and restructuring.

Their individual actions, when combined, result in synergistic effects that optimize the healing process.

By incorporating these products into wound care protocols, healthcare providers and individuals can benefit from their outstanding properties and achieve superior healing outcomes.

Given the promising results and the growing interest in the combination of honey, trigonelline, and zinc oxide, it is evident that innovation and technology development in this field will continue to grow and evolve, focus on enhancing the efficacy, safety, and quality of products related to this combination.

While many unanswered questions remain, the composition offers hope to patients with damaged or diseased tissue, for whom the possibilities for repair-regeneration and restructuring are virtually endless.

Continued research and clinical investigations will further validate the efficacy and safety of integrating honey, trigonelline, and zinc oxide in wound healing.

In addition, long-term clinical trials are needed to assess the sustained benefits and therapeutic effects in combination with conventional therapies, leading to more efficient wound restructuring, reduced healing time, and better patient outcomes.

### BRIEF DESCRIPTION OF THE SEVERAL VIEWS OF THE DRAWING

Not Applicable

### SUMMARY OF THE INVENTION

Chronic wounds present substantial obstacles within the realm of wound healing, necessitating comprehensive strategies that target the fundamental cellular, molecular, and local elements contributing to their resolution.

To promote a harmonized and well-coordinated healing response, it becomes imperative to offer a comprehensive multi-component therapeutic solution that effectively challenges inflammation, infection, oxidation, immune system deficiencies, cellular and molecular mechanisms, angiogenesis, granulation tissue formation, and the dynamics of the extracellular matrix (ECM).

The multifunctional composition for wound healing, obtained from bee honey, trigonelline (N-methylnicotinic acid) seed extract and zinc oxide, shows various bioactivities that include antibacterial, anti-inflammatory, antioxidant, and immunomodulatory properties, making it an effective alternative for the management of various skin conditions.

By integrating into the composition, the three products that actively resolve infections, the modulation of inflammation and the immune system, the reactivation of ECM, the optimization of the tissue microenvironment, the restoration of cellular and molecular mechanisms, the promotion of angiogenesis, the improvement of the formation of granulation tissue and epithelialization, complete healing results are obtained.

### DETAILED DESCRIPTION OF THE INVENTION

While the present invention is described with respect to what is presently considered to be the preferred aspects, it is to be understood that the invention as claimed is not limited to the disclosed aspects.

Furthermore, it is understood that this invention is not limited to the particular methodology, materials and modifications described and as such may, of course, vary.

It is also to be understood that the terminology used herein is for describing particular embodiments only, and is not intended to be limiting, since the scope of the present invention will be limited only by the appended claims.

Unless otherwise defined, all technical and scientific terms used herein have the same meaning as is commonly understood by those skilled in the art to which this invention pertains.

The present invention for promoting or expedite tissue repair, regeneration, and restructuring of damaged skin tissues, provides an example of a wound healing composition, including the terms:

Angiogenesis—the physiological process of forming new blood vessels from pre-existing ones.

Antibacterial—substances or agents that can inhibit the growth or killing bacteria.

Anti-inflammatory—substances or treatments that reduce inflammation.

Bacterial—microorganisms that belong to the domain Bacteria.

Chronic wound—a type of wound that fails to progress through the normal stages of healing within an expected timeframe.

Connective tissue—a type of tissue that provides support, structure, and connection between various organs and tissues in the body.

Debridement—the process of removing dead, damaged, or infected tissue from a wound to facilitate healing.

Dressing—a material or product applied to a wound to protect it and promote healing.

Dermis—the layer of skin located beneath the epidermis, involved in providing structural support and nourishment to the skin.

Disease—any condition or disorder that causes damage or disruption to the normal functioning of a cell, tissue, or organ.

Effective amount—the quantity of an agent necessary to alleviate the symptoms of a disease compared to a patient who remains untreated.

Enzymatic activity—the capability of enzymes to catalyze specific biochemical reactions in the body.

Epidermis—the outermost layer of the skin.

Epithelialization—the process of new skin cells migrating and proliferating to resurface a wound.

Excision—the surgical removal damaged or diseased tissue.

Extracellular matrix—a network of extracellular macromolecules and minerals, that provide structural and biochemical support to surrounding cells.

Granulation tissue—a type of tissue that forms during the proliferative phase of wound healing, consists of new blood vessels, fibroblasts, and extracellular matrix components.

Infection—the presence of one or more pathogens infiltrating the tissues or organs of a host organism.

Immunomodulator—a substance or treatment that can modify or regulate the immune response of an organism.

Microenvironment—the local cellular and molecular environment surrounding cells in a specific tissue or organ.

Modulation—any alteration, whether it be an increase or decrease, in the function or activity of a specific biological process.

Necrosis—the death of cells or tissues due to various factors, such as injury, infection, or inadequate blood supply.

Obtaining—a range of processes such as synthesis, procurement, or any other means of acquiring the desired agent.

Oxidation—a chemical reaction that involves the loss of electrons or an increase in oxidation state.

pH—the acidity or alkalinity of a solution.

Prevent, preventing, prevention—aim to convey the act of diminishing the probability of an individual developing a disorder or condition.

Reconstruction—defined as “replacing and rebuilding what is torn down.

Repair—refer to the reestablishment of tissue continuity.

Regeneration—refer to the complete replacement of damaged tissue with new tissue not associated with scar tissue.

Restructuring—the process of rearranging or rebuilding the structure of a tissue or organ.

Ripe honey—honey that has been collected from beehives and ripened through the bees' enzymatic and evaporative processes.

Small molecule—any chemical compound, regardless of its molecular weight or structural complexity, emphasizing inclusivity within its definition.

Subject—denotes a mammal, embracing a wide spectrum that encompasses humans as well as non-human mammals such as bovines, equines, canines, ovine, or felines.

Sterilization—the process of eliminating or destroying all forms of microbial life, including bacteria, viruses, and fungi.

Stromal tissue—the supportive, non-epithelial component of a tissue or organ.

Trauma—any form of injury that inflicts damage upon the tissues or organs of a subject.

Wound dressing—material or product applied directly to a wound to protect it, promote healing, and maintain a favorable environment for wound closure.

The invention is directed to a multifunctional vulnerary composition comprising honey with anti-inflammatory, antimicrobial, and wound healing properties, trigonelline extract with anti-inflammatory, antioxidant, and collagen-stimulating effects, and zinc oxide with antimicrobial, wound healing, and skin barrier protection properties, methods of producing the same, and therapeutic applications arising from their utilization.

In a first embodiment, the use of appropriate wound care composition and interventions, can substantially improve clinical outcomes and reduce unnecessary morbidity and mortality, in chronic injuries.

In a specific embodiment, the new technology refers to further improvements in subject care and better procedures involved in surgical wound repair-regeneration and restructuring.

In a second embodiment, the present disclosure, is directed to a new multi-functional composition for promote or expedite the repair-regeneration and restructuring of mammals' skin tissues.

In another embodiment, the present invention introduces a multifunctional vulnerary composition comprising honey with anti-inflammatory, antimicrobial, and wound healing properties, trigonelline extract with anti-inflammatory, antioxidant, and collagen-stimulating effects, and zinc oxide with antimicrobial, wound healing, and skin barrier protection properties.

This composition represents a groundbreaking approach to promote tissue repair, regeneration, and restructuring solution for damaged mammals skin tissues.

According to a specific embodiment, the invention discloses a multifunctional vulnerary composition specifically formulated for promoting tissue repair, regeneration, and restructuring of damaged mammals skin tissues.

By incorporating honey with anti-inflammatory, antimicrobial, and wound healing properties, trigonelline extract with anti-inflammatory, antioxidant, and collagen-stimulating effects, and zinc oxide with antimicrobial, wound healing, and skin barrier protection properties, this composition provides a synergistic and comprehensive approach to address damaged mammals skin tissues.

In another embodiment, the invention emphasizes a multifunctional vulnerary composition comprising honey, fenugreek extract, and zinc oxide, each known for their unique properties to promote tissue repair, regeneration, and restructuring.

Through honey's anti-inflammatory, antimicrobial, and wound healing properties, trigonelline extract's anti-inflammatory, antioxidant, and collagen-stimulating effects, and zinc oxide's antimicrobial, wound healing, and skin barrier protection properties, this composition represents an innovative solution for damaged mammals skin tissues.

Another embodiment of the invention highlights the advance of a multifunctional vulnerary composition comprising honey present in a concentration range of 5-30% by weight, wherein the trigonelline extract is present in a concentration range of 3-15% by weight, wherein the zinc oxide is present in a concentration range of 2-20% by weight and wherein the excipients are present in a concentration range of 45-55% by weight.

Another embodiment of the invention the honey present in a concentration range preferably of 15-22% by weight, trigonelline extract present in a concentration range preferably of 2-8% by weight, zinc oxide present in a concentration range preferably of 10-17% by weight and excipients accounting for 54.4% by weight including a pH-modifying constituent in an amount sufficient to adjust the Ph to a value of 3.5-5.

Moreover, the composition incorporates excipients accounting for 54.4% by weight including a PH-modifying constituent in an amount sufficient to adjust the PH to a value of 3.5-5, offering an accurately formulation for promoting tissue repair, regeneration, and restructuring.

In another embodiment, this invention presents an advanced methodology where the harmonized action of bee honey, trigonelline extract and zinc oxide synergistically contribute to reducing inflammation, controlling bacterial load, cell protection against oxidative stress, modulation of the immune response, and repair-regeneration and restructuring of mammals damaged tissues.

In accordance with one embodiment, this invention provides a formulation for simultaneous treatment of inflammation, infection, oxidative damage, and immunological compromise in wounds, using a new multifunctional vulnerary composition that incorporates honey, trigonelline extract and zinc oxide.

In accordance with other embodiment, this invention introduces an advanced methodology where the harmonized action of bee honey, trigonelline extract, and zinc oxide plays a pivotal role in accomplishing various therapeutic objectives, including inflammation reduction, bacterial weight control, cellular protection against oxidative stress, immune response modulation, and mammals tissue repair-regeneration and restructuring.

In another embodiment, the invention encompasses a composition for modulating the inflammatory response during wound healing.

Another embodiment of the invention relates to a composition for reducing the risk of infection during wound healing.

Another embodiment of the invention relates to a composition for reducing oxidative stress in biological systems.

According to a specific embodiment, the present invention discloses a composition for the delivery of therapeutic metabolites to modulate the immune response, at the wound site.

In one embodiment, the invention discloses a system for addressing local wound bed problems, including angiogenesis, granulation tissue formation, and epithelialization.

Another embodiment of the invention relates to a composition for promoting epithelialization and accelerating wound closure.

In another embodiment, the present invention discloses a composition that enhances the restructuring of the extracellular matrix (ECM), promoting improved tissue repair and regeneration.

Another embodiment of the invention encompasses a composition capable of reducing hypertrophic scarring during wound healing and providing improved functional and aesthetic outcomes.

According to a specific embodiment, the invention provides a method for formulating topical preparations, including pastes, creams, lotions, serums, gels, hydrogels, hydrocolloids, and films to facilitate easy application and effective delivery of natural products to mammals damaged skin tissues, promoting enhanced tissue repair, regeneration, and restructuring.

While the compositions and methods of this invention have been described in terms of preferred embodiments, it will be apparent to those of skill in the art that variations may be applied to the methods and in the steps or in the sequence of steps of the methods described herein without departing from the concept, spirit and scope of the invention.

The following examples are included to demonstrate preferred embodiments of the disclosure.

It should be appreciated by those of skill in the art that the techniques disclosed in the examples which follow represent techniques discovered by the inventor to function well in the practice of the invention, and thus can be considered to constitute preferred modes for its practice.

However, those of skill in the art should, considering the present disclosure, appreciate that many changes can be made in the specific embodiments which are disclosed and still obtain a like or similar result without departing from the spirit and scope of the invention.

While the fundamental novel features of the tissue disclosed herein have been described, it will be understood that various omissions, substitutions and changes in the form and details may be possible without departing from the spirit of the present disclosure.

### Example #1

### Product Manufacturing Methods

The composition extraction method, includes the following steps:
a) Ultrasonic extraction of the Trigonelline Extract, and then filtration.
b) In a kettle add excipients, with lubrication functions (10 to 30%), emulsifiers (1%-12%), softener (3% to 25%), preservative (0.1%-2%), absorbent (0.1%-5%).
c) Zinc oxide (2%-20%), and purified water as vehicle (3%-50%), then heat to 80° C. and mix until incorporated.
d) Add the following raw materials to the previous mixture: honey (5%-30%) and trigonelline Extract (3%-15%) until temperatures between 30 to 60° centigrade are reached. shaking constantly.
e) Add the excipients with Antioxidant (0.1%-3%) and Regenerator (0.1%-3%) functions to the previous mixture, with constant stirring.
f) Adjust the pH, if necessary, with a pH buffer solution (0.001%-1%).

### Example #2

Indications for use: The multifunctional vulnerary composition may be used in both the human and veterinary field, for temporary coverage of superficial and deep second-degree burn (partial thickness burn), pressure ulcers, leg ulcers, surgical wounds, trauma wounds (abrasions, lacerations, erosions), and donor sites.

### Preventive Aspect

Prior to the placement of the multifunctional vulnerary composition, it is necessary to perform several actions, due to the impact they have on the healing of wounds:

Complete evaluation of the patient (include comprehensive vascular evaluation).

Determining the etiology of wound (i.e., acute, chronic, ischemic). Ascertain the underlying disease, e.g., venous/arterial.

Use a validated pressure ulcer classification system to document the level of tissue loss (i.e., Braden Scale, Norton Scale, Waterlow Score Card, Braden Q (for pediatric patients), or other appropriate tool in conjunction with clinical judgment.

Utilize appropriate and regular measurement of wound size (take a tracing of the wounds).

Determining the bacterial bioburden of the injury by tissue biopsy or quantitative swab technique (i.e., Levine quantitative swab technique).

Sharp debridement and antibiotic therapy must be initiated as early as possible, and the blood glucose should be monitored closely and controlled.

Assess for osteomyelitis or skin neoplasms in chronic ulcers with appropriate Laboratory tests (biopsy of every non-healing wound) and x-rays as need.

Nutritional assessment and lab evaluation of plasma proteins and Hematocrit-Hemoglobin.

Such information it is essential to identify these factors and control them to facilitate faster wound healing whenever possible.

Placement Procedure of the multifunctional vulnerary composition

Prepare wounds very meticulous, to guarantee lesion is entirely excised or surgically debrided, to ensure the wound bed and edges contain viable structures, free of debris and necrotic tissue.

With normal saline solution gently irrigated the injury.

Debride blister and loose skin.

Cleanse with warmed normal saline.

Avoid broad area of capillary bleeding which can lead to devitalized tissue.

To apply the paste (the preferred method of composition presentation):

Cleanse the wound with a saline or mild antiseptic solution.

Gently apply a thin layer of the honey, fenugreek, and oxide paste directly onto the wound bed.

Repeat the application, depending on the severity of the wound, although in most cases it is done every 5 to 7 days, except in Diaper Dermatitis lesions, a situation in which it must be applied with each change of this infant garment.

Cover the wound with a sterile dressing to protect it from external contaminants.

Dressings to protect practical active dressing as follows:

High quality white nylon mesh and a tubular elastic bandage applied over the composition to facilitate its permanence and protect the site.

Both can be left in place for extended periods without detrimental effects to the underlying wound.

In this sense, preparing Bowl with sterile saline solution and immersing the nylon mesh (second dressing) for 10 seconds.

Next the wound bed with the composition is overlaid with this material in direct contact with wound surface and then bandaged with elastic tubular dressing (third dressing) to preserve adherence and permit easy inspections.

Careful inspection of the wound site and the clinical monitoring of patients' conditions allow the clinician to prolong the interval between dressing changes.

It is the opinion of the author that a maximum of one continuous week without a dressing change is acceptable if no signs of infection develop.

At seven or more days, the bandage can be removed with gentle saline irrigation or showering.

Do not forcibly remove sections of the bandage that have adhered to the wound.

Subsequently, with new watering, they are completely removed.

Complications: The following complications are possible with the use of any wound dressings.

If any of the conditions occur, the composition should be removed:

infection, inflammation, allergic reaction, excessive redness, pain or swelling.

Complications that the surgeon may encounter further than infection include seroma and/or hematoma formation.

Although wound infection is rare, when this is suspected, appropriate bacterial identification is obligatory and according to the cultivated microorganisms' antibiotics should be prescribed.

### Example #3

The present invention closely conforms to the progress of a previously studied dressing, referring to the International Patent Publications No. WO 2014041440 A1 Publication date Mar. 20, 2014^{[8]} and Multifunctional-reinforced dressing Inventor Carlos A. Alvarado 2018 Feb. 8, Publication of WO 2018025060 A1^{[9]} earlier prepared by the inventor, whose variations give rise to the present invention.

Comparative clinical study to assess the efficacy and safety of new multifunctional dressing.

### Introduction

The therapeutic approach and intervention in the formation of granulation tissue in difficult to healing ulcers, suppose provide safe and quality care to people affected by these conditions.

This implies the need to investigate what may be the most effective possible protocols to guide and direct the actions of care for such chronic and complex lesions, in order to improve the quality of life of patients and incorporate in the country advanced and effective technology with the latest scientific knowledge and solution at a low cost.

### Objective

To compare the clinical results of treatment with traditional cures and advanced treatments with multifunctional dressing in hard-to-heal wound.

### Material and Methods

The development and evaluation of a randomized trial with two treatment groups for patients with difficult to cure ulcers, one to which with a multifunctional dressing with cures every 3 days and another with wet-to-dry with gauze dressings and saline changed daily.

The study population is composed 380 adults treated in the Outpatient Department of Surgery at Roosevelt Hospital in Guatemala City in the period from January to October of 2,014, with Protocols that had been reviewed and approved by ethics committee.

Patients must meet the eligibility criteria as: both sexes, over 18 years, presence of ulceration in the lower limbs with a maximum diameter of 150 cm 2 and equal to or greater than 90 days of evolution.

The study included eligible subjects, according to the criteria of inclusion and exclusion, with individual clarification to the patient and family about the goals and methods of the study and was provided informed consent form, which included reading, understanding and signature for inclusion in the investigation.

Treatments were performed with the multifunctional dressing in pairs new cases and in wet to dry no pairs cases, with Primary outcomes in relation to the formation of granulation tissue and/or epithelial tissue in the wound bed, with collection data measurement in 4 times (at baseline, four, eight and twelve weeks).

For the assessment of characteristics at baseline and evolution of treatment of injuries, a modified and reduced table the report of Houghton et al, which included visual evaluation and measurement of five components was developed: size, type and amount of necrotic tissue, type, and amount of granulation tissue.

A number from 0 to 4 was assigned to each of them, then count the five components to get the total score of the wound, with the possible total scores range from 0 to 24, with zero representing a completely healed ulcer.

Reliability, and sensitivity was determined by the indicated visual assessments and ruler measurements (maximum and maximum width of the wound length), by a single evaluator during the study period.

For the safety, were included assessment of symptoms reported by patients and clinical signs observed by the physician.

### Results

380 patients were included in the evaluation (190 in each group studied), of which 61% were female and 39% male.

Of this number only 153 patients completed the study (81%) of the multifunctional dressing group and 98 cases (52%) of the Wet-to-dry and gauze dressings with a pronounced treatment discontinuation in the first four weeks in both groups.

The greatest treatment discontinuation was particularly due to lack of patient compliance to healing, which was most evident in the treatment group Wet-to-dry and gauze dressings.

On average 3 weekly cures for the multifunctional dressing and 10 weekly cures for the Wet-to-dry and gauze dressings were performed.

It was determined that in 203 patients with ulcerations were diabetic patients (54%), and ulcerations due to burns, soft tissue infections in 96 cases (25%), occupying venous insufficiency third place with 54 patients (14%) and arterial disease with 27 cases (7%).

The lesions were present in the foot in 53% of cases, in the leg in 23% of them, in the sacral region in 16% of them, and trochanteric region at 8%.

Regarding the time evolution of lesions in 52% of cases ulceration was present between three to six months and the remaining 48% of cases over six months.

At the first evaluation at four weeks, it was shown that the ulcerations that were treated with the multifunctional dressing had progressed adequately to healing with intact skin or in close proximity to, in 37% of cases compared with 11% the wet dry cures.

When making the second assessment at eight weeks, it was shown that the ulcerations that were treated with the multifunctional dressing had progressed to healing with intact or in close proximity to, in 50% of cases, in contrast to the progress of 20% the wet dry cures.

When performing the third evaluation at twelve weeks (end), it was shown that the ulcerations that were treated with the multifunctional dressing had progressed to healing with intact skin or in close proximity to, at 64% % of cases, in contrast to the progress of 25% of the wet dry cures.

### Conclusions

The results of a study showed that the multifunctional dressing leads to improved and accelerated tissue repair-regeneration in chronic ulcers from healing compared to healing method from the Wet-to-dry and gauze dressings.

### INDUSTRIAL APPLICATIONS

The composition, based on three key ingredients (bee honey, trigonelline (N-methylnicotinic acid), and zinc oxide), offers several significant advantages, particularly due to its cost-effectiveness, ease of production, and simple application to wounds.

These characteristics make it highly suitable for economically disadvantaged regions, where accessibility to medical resources might be limited.

The advancement of manufacturing technology is anticipated to become the primary approach for developing new products in various industrial processes and applications.

Moreover, these ingredients are derived from sustainable and easily accessible sources, enabling the mass production of this composition in a consistent and profitable manner.

This ensures a reliable supply of the product, further contributing to its potential impact on wound care in underserved areas.

From a social perspective the product is accessible, reasonably, for the benefit of the population who are economically disadvantaged and, in the future, may need it.

For developing countries, consider concerns of “access” to technology. The present invention addresses this need.

In summary: technology improves the lives of hundreds of thousands of patients and solves a major problem for the less developed areas of the world by providing an economic product.

### Patent Citations (9)

| Publication | Priority | Publication | | |
|---|---|---|---|---|
| Number | Date | Date | Assignee | Title |
| WO2012134770A1 * | 2011 Mar. 25 | 2012 Oct. 4 | Avery | Honey |
| | | | Dennison | composition |
| | | | Corporation | |
| WO2014159419A1 * | 2013 Mar. 14 | 2014 Oct. 2 | 3M Innovative | Hydrocolloid |
| | | | Properties | wound dressings |
| | | | Company | with increased |
| | | | | wvtr |
| JP2003516357A * | 1999 Dec. 9 | 2003 May 13 | [symbol] | Honey-based medical dressing |
| | | | (Waikato Link | |
| | | | Limited) | |
| WO 2014041440 Al | 2013 Feb. 5 | 2014 Mar. 20 | Alvarado | Honey-based |
| | | | Carlos A. | dressing for the |
| | | | | treatment of |
| | | | | wounds and |
| | | | | burns |
| US 20150056175 A1 | 2013 Mar. 16 | 2015 Feb. 26 | García | Composition for |
| | | | Gilabert | preventing |
| | | | | and/or treating |
| | | | | dermatosis and |
| | | | | method for |
| | | | | obtaining same. |
| WO2018025060A1 | 2016 Aug. 1 | 2018 Feb. 8 | Alvarado | Multifunctional- |
| | | | Carlos A. | reinforced |
| | | | | dressing |
| US20160220722A1 | 2016 Jan. 29 | 2019 Dec. 10 | Wardell M. | Wound Healing |
| | | | and Sabacinski | Compositions |
| | | | K. | Involving |
| | | | | Medicinal |
| | | | | Honey, Mineral |
| | | | | ions, and |
| | | | | Methylglyoxal, |
| | | | | and Methods of |
| | | | | Use |
| US20180236009A1 | 2017 Oct. 20 | 2018 Aug. 23 | Sabacinski K. | Buckwheat |
| | | | And Kaufman | honey and |
| | | | J. L. | bacitracin |
| | | | | wound-healing |
| | | | | dressing. |
| WO2019040185A1 | 2018 Jun. 29 | 2019 Feb. 28 | Sabacinski K. | Buckwheat |
| | | | and Kaufman | honey and |
| | | | J. L. | povidone-iodine |
| | | | | wound-healing |
| | | | | dressing. |

### Non-Patent Citations (23)

[1] Ingawale D K, Mandlik S K, Patel S S. An emphasis on molecular mechanisms of anti-inflammatory effects and glucocorticoid resistance. J Complement Integr Med. 2015; 12 (1): 1-13. doi: 10.1515/jcim-2014-0051.
[2] Erejuwa O O, Sulaiman S A, Ab Wahab M S. Effects of honey and its mechanisms of action on the development and progression of cancer. Molecules. 2014; 19 (2): 2497-2522. doi: 10.3390/molecules19022497.
[3], Wu, W., Jin, Y., Fang, B., and Jin, S. (2015). “Chapter 41-Pseudomonas 10 aeruginosa,” in Molecular Medical Microbiology, eds Tang Y-W and A. Sails (Amsterdam: Academic Press).
[4] Azam, M., and Khan, A. U. (2019). Updates on the pathogenicity status of Pseudomonas aeruginosa. Drug Discov. Today 24, 350-359. doi: 10.1016/j.drudis.2018.07.003
[5] Kirchner S, Lei V, Macleod A S. The Cutaneous Wound Innate Immunological Microenvironment. Int J Mol Sci. 2020 Nov. 19; 21 (22): 8748. doi: 10.3390/ijms21228748. PMID: 33228152; PMCID: PMC7699544.
[6] Berkes, E and Monsul T. (2016). Materials and Methods for Treating Conditions Associated with Pathogenic Biofilm. (U.S. Patent No. US-20140037688-A1). U.S. Patent and Trademark Office.
[7] Djoko, K. Y., Ong, C. L. Y., Walker, M. J., and Mcewan, A. G. (2015). The role of copper and zinc toxicity in innate immune defense against bacterial pathogens. J. Biol. Chem. 290, 18954-18961. doi: 10.1074/jbc. R115.647099
[8] Alvarado Carlos A. (2014). Honey-based dressing for the treatment of wounds and burns. (WO 2014041440 A1). WIPO (PCT).
[9] Alvarado Carlos A. (2018). Multifunctional-reinforced dressing. (WO 2018025060 A1). WIPO (PCT).
[10] Tashkandi H. Honey in wound healing: An updated review. Open Life Sci. 2021 Oct. 6; 16 (1): 1091-1100. doi: 10.1515/biol-2021-0084. PMID: 34708153; PMCID: PMC8496555.
[11] Almasaudi S B, El-Shitany N A, Abbas A T, Abdel-dayem U A, Ali S S, A1 Jaouni S K, et al. Antioxidant, anti-inflammatory, and antiulcer potential of manuka honey against gastric ulcer in rats. Oxid Med Cell Longev. 2016; 2016:3643824.
[12] Molan P C. Re-introducing honey in the management of wounds and ulcers—theory and practice. Ostomy Wound Manage. 2002; 48 (11): 28-40.
[13] Molan P C. The evidence supporting the use of honey as a wound dressing. Int J Low Extrem Wounds. 2006; 5 (1): 40-54.
[14] Molan P C, White R. Honey in modern wound management. Aberdeen, U K: Wounds U K Ltd; 2009.
[15] Scepankova H, Combarros-Fuertes P, Fresno J M, Tornadijo M E, Dias M S, Pinto C A, Saraiva J A, Estevinho L M. Role of Honey in Advanced Wound Care. Molecules. 2021 Aug. 7; 26 (16): 4784. Doi: 10.3390/molecules26164784. PMID: 34443372; PMCID: PMC8398244.
[16] Imran, F.-H.; Dorai, A. A.; Halim, A. S.; Sulaiman, W. A. W. Tualang honey hydrogel in the treatment of split-skin graft donor sites. J. ApiProd. ApiMed. Sci. 2011, 3, 33-37.
[17] Garcia Gilabert, J. M. (2015). Composition for preventing and/or treating dermatosis and method for obtaining same. (U.S. Patent No. US20150056175-A1). U.S. Patent and Trademark Office.
[18] Chang Espino, M. J., Navas Escobedo I., Evaluación del efecto cicatrizante de semillas de Trigonella foenum-graecum L. (fenogreco) y hojas de Plantago major L. (llantén) en heridas producidas en ratas albinas. Tesis (Químico Farmacéutico) Facultad de CC. QQ. y Farmacia, USAC. Guatemala: USAC, 1992.
[19] M. R. Mustafa, A. A. Mahmood, and I. Salmah, 2005. Effects of Trigonella foenum graecum Seed Extract in Combination with Honey on Experimental Wound Healing in Rats. International Journal of Molecular Medicine and Advance Sciences, 1:29-33.
[20] Lansdown A B, Mirastschijski U, Stubbs N, Scanlon E, Agren M S. Zinc in wound healing: theoretical, experimental, and clinical aspects. Wound Repair Regen. 2007 January-February; 15 (1): 2-16. doi: 10.1111/j.1524-475X.2006.00179. x. PMID: 17244314.
[21] Ravanti L, Kähäri V M. Matrix metalloproteinases in wound repair (review). Int J Mol Med 2000; 6:391-407.

1- A composition for promoting or expedite tissue repair, regeneration, and restructuring of damaged skin tissues in mammals, which is characterized by comprising:
a) Honey of bee
b) Trigonelline extract
c) Zinc oxide, and
d) Excipients

2- The composition of claim 1, wherein the honey is present in a concentration range of 5-30% by weight, wherein the trigonelline extract is present in a concentration range of 3-15% by weight, wherein the zinc oxide is present in a concentration range of 2-20% by weight and wherein the excipients are present in a concentration range of 45-55% by weight.

3- The composition of claim 1, wherein the honey present in a concentration range preferably of 15-22% by weight, trigonelline extract present in a concentration range preferably of 2-8% by weight, zinc oxide present in a concentration range preferably of 10-17% by weight and excipients accounting for 54.4% by weight including a pH-modifying constituent in an amount sufficient to adjust the Ph to a value of 3.5-5.

4- The composition of claim 1, wherein the composition is formulated into topical preparations, including pastes, creams, lotions, serums, gels, hydrogels, hydrocolloids, and films.

5- A method of preparing a composition for promoting or expeditious tissue repair, regeneration, and restructuring of damaged skin tissues in mammals, which is characterized by the following steps:
a) Ultrasonic extraction of the Trigonelline Extract, and then filtration.
b) In a kettle add excipients, with lubrication functions (10 to 30%), emulsifiers (1%-12%), softener (3% to 25%), preservative (0.1%-2%), absorbent (0.1%-5%).
c) Zinc oxide (2%-20%), and purified water as vehicle (3%-50%), then heat to 80° C. and mix until incorporated.
d) Add the following raw materials to the previous mixture: honey (5%-30%) and trigonelline Extract (3%-15%) until temperatures between 30 to 60° centigrade are reached. shaking constantly.
e) Add the excipients with Antioxidant (0.1%-3%) and Regenerator (0.1%-3%) functions to the previous mixture, with constant stirring.
f) Adjust the pH, if necessary, with a pH buffer solution (0.001%-1%).

6- A method for promoting or expedite tissue repair, regeneration, and restructuring of damaged skin tissues in mammal, said method comprising: topical application of the multifunctional vulnerary composition, in any presentation of the formulation: pastes, creams, lotions, serums, gels, hydrogels, hydrocolloids or films in direct contact with defective skin surfaces.
